(19) United States
(12) Patent Application Publication
(10) Pub. No.: US 2003/0198696 A1
(43) Pub. Date: Oct. 23, 2003

(54) Pesticide composition of plant oils and fish oils

(76) Inventor: David Keen, Stuart, FL (US)

Correspondence Address: OLSON & HIERL, LTD., 36th Floor, 20 North Wacker Drive, Chicago, IL, 60606, US

(21) Appl. No.: 10/310,110

(22) Filed: Dec. 4, 2002

Related U.S. Application Data
(63) Continuation of application No. 09/691,066, filed on Oct. 19, 2000, now abandoned.

(51) Int. Cl. A61K 35/78; A61K 35/60

(52) U.S. Cl. 424/725; 424/523

(57) ABSTRACT

This invention pertains to a pesticide composition effective for the control of insects and diseases of plants in a synergistic formulation of plant oils and fish oils that is non-toxic to the plant, the environment and to those applying the pesticide.

### FIELD OF THE INVENTION

This invention relates to a pesticide composition of natural components found in nature, namely, oils derived from plants and fish or mixtures thereof that act as the active ingredients. The oils may or may not be considered “essential oils.” As used herein, reference to “essential oil” should be understood as defined in the text by Ernest Guenther, The Essential Oils, (1972) R. E. Krieger Publishing Company, Huntington, N. Y. “Plant oils” should be understood to mean any plant oil other than essential oil.

### BACKGROUND OF THE INVENTION

“Pesticide” is defined herein in its broadest sense, Webster's Third New International Dictionary, copyright 1961, defines “pest” as “an epidemic disease,” or “anything resembling a pest in destructiveness, noxiousness, etc.” Accordingly, a “pesticide” is an agent used to destroy a pest or to kill insects, bacterial, fungi and other forms of undesirable plant or animal life.

Use of synthetic pesticides has proven to be very effective in protecting crops from insects and diseases. However, synthetic pesticides can be toxic to the environment and to those applying the pesticide. A concern for the toxic effects of synthetic pesticides on the environment and those in contact with synthetic pesticides has prompted the development of a safer, natural pesticide that poses no risk to the environment or to those applying the pesticide. Also certain plant essential oils such as citrus oil or limonene by themselves can be toxic to plants at rates necessary to kill or control insects and disease. By combining plant essential oils such as citrus oil or limonene with fish oil the phytotoxic effects of those oils are greatly reduced or eliminated.

While the use of naturally occurring, or so-called “soft” pesticides is desirable to replace synthetic or “hard” pesticides, the effectiveness of soft pesticides has left a lot to be desired in controlling insects and diseases on plants. The ability of “soft” pesticides to satisfactorily replace “hard” pesticides has been extremely limited and for the most part a failure. In most cases the hard pesticide is superior to the soft pesticide. Often, the targeted insects and diseases are not effected by softer pesticides, and plants or crops are damaged for lack of adequate insect and disease control.

This invention can be classed as a soft pesticide derived from naturally occurring components. Surprisingly and unexpectedly this soft pesticide performs as well or better than the synthetic pesticide it replaces. It is also surprising the phytotoxicity of certain plant essential oils such as citrus oil or limonene when used as an active ingredient is greatly reduced or eliminated when combined with fish oils.

The pesticide composition may also contain surfactants or emulsifiers that may or may not be natural substances and act as the inactive ingredient. The composition is in a form that is not toxic to the plant.

The invention may also be combined with other pesticides for a synergistic effect on insect and disease control. The active ingredients of this invention combined with other types of pesticides allows for a reduction in the use rate of other pesticides without any loss of effectiveness. This is particularly important in reducing the overall amount of pesticides used in the environment.

The plant oils and essential oils may be derived from but not limited to the following plants selected from the group consisting of: castor, cedar, cinnamon, citronella, citrus, clove, coconut, corn, cottonseed, garlic, geranium, lemon grass, linseed, mint, neem, olive, oregano, palmarosa, peanut, peppermint, rape, rosemary, sesame, soybean, tea tree and thyme or mixtures thereof.

The fish oils may be derived from but not limited to fish selected from the group consisting of: cod, haddock, capelin, squid, hake, shark, halibut, menhaden, sardine, herring, pollack, cuttle, mackerel, sand eel, anchovy, salmon and gadoid or mixtures thereof.

Examples of pesticides which can be combined with this invention include, but not limited to, are fungicides, herbicides, insecticides, miticides, bactericides, nematicides, algacides and biological pesticides. Those skilled in the art will know the many types and classes of pesticides available.

The prior art teaches there are many natural substances that can be used to protect plants from insects and diseases. For example, U.S. Pat. No. 2,198,991 discloses a method for protecting living trees and plants from sunscald, borer and fungus injury by treating the trunks and branches with an aqueous emulsion comprising a paraffin wax and an ammonium salt of a drying acid.

U.S. Pat. No. 4,826,863 disclose the use of various oils such as, paraffin, soy, fish and mineral oils, together with, inter alia, the active ingredient such as fungicide or herbicide, to provide a stabilized plant protecting suspension. The inventor teaches the natural oils are used as an inactive component for the stabilization of inorganic or synthetic active ingredients in the composition. No pesticide claims are made for the natural oils.

U.S. Pat. No. 4,734,432 discloses the use of a vegetable, animal or mineral oil together with, inter alia, a fungicide or insecticide. Again, the inventor is using a natural oil component as an inactive ingredient to impart suspension capabilities for an inorganic or synthetic active ingredient.

U.S. Pat. No. 5,110,591 discloses a concentrated water-in-oil neem oil emulsion which upon dilution with water, form storage stable oil-in-water emulsions. The inventor has disclosed a formulation of concentrated water-in-oil microemulsions containing neem oil. The inventor teaches the concentrated neem oil emulsion must contain a certain amount of water in order to form a storage stable emulsion. My invention differs in that neem oil is soluble in the fish oil and water is not needed to form a storage stable pesticide composition.

U.S. Pat. No. 5,720,980 teaches the use of fish oil, preferably with a metal salt to protect a crop against fungal diseases. The inventor does not claim the merits of a combination of plant and fish oils nor does the invention make any claims for controlling both insects and disease pests.

U.S. Pat. No. 4,442,092 discloses a nematocidal composition containing extracts of the sesame plant including sesame oil. Again the inventor does not claim the merits of a combination of plant and fish oils nor does the inventor make any claims for controlling both insects and disease pests. This invention is narrow in scope as it pertains only to the control of a single pest.

U.S. Pat. No. 5,658,954 discloses an environmental insecticide using plant oil as the starting raw materials and converting them to amides such as cottonseed oil amides, coconut oil amides, tall oil amides. The insecticidal oil-containing amide is a reaction product of a fatty acid or a fatty acid ester and amine, such as monoethanolamine. Therefore the active ingredients of the invention cannot be considered natural as in my present invention.

U.S. Pat. No. 4,885,177 teaches the use of soybean meal and fish meal that may or may not be combined with a chemical fertilizer for soil treatment to control the corn rootworm. The inventor acknowledges he doesn't know whey his invention works and that the active ingredients in these meals may not be derived from their respective oils but instead from the meals themselves. In no case did the inventor utilize the plant and fish oils to assess their combination insect and disease protection capability.

U.S. Pat. No. 4,681,617 discloses the use of a phospholipid composition in plant protection spray mixtures. The inventor acknowledges that phospholipid composition is not the actual active ingredient but acts as an adjuvant for improving the performance of pesticides.

U.S. Pat. No. 4,599,233 discloses composition of sodium bicarbonate and lecithin for the control of certain fungal diseases in plants. My invention does not use any mineral component as an active ingredient.

U.S. Pat. Nos. 3,728,454, 3,725,557, and 3,728,453 disclose the use of pine oil and fish oil, together with, inter alia, the active ingredient, alloxan or alloxantin, or dialuric acid, respectively, to inhibit the growth of herbs, bacteria, fungi and other microorganisms. The inventor claims the plant and fish oils as an inactive ingredient and furthermore, makes no claim to both insect and disease control.

The prior art did not recognize the potency of a natural pesticide composition of plant and fish oil mixtures and the broad range of control this composition has on both insects and diseases of plants. The prior art also did not recognize that fish oil greatly reduces or eliminates the toxic effects on plants of certain plant oils such as essential oils.

In accordance with the present invention, it has been found that plant oil, essential oil and fish oil or mixtures thereof provides an extremely potent natural pesticide for the control of plant insect pests such as, aphids, chinch bugs, citrus rust mites, spider mites, armored and soft scales, mealy bugs, white-flies, leaf-rollers, leaf miner, fungus gnats, nematodes, sharp-shooters and thrips. It has been found to be effective on all life stages of insects including eggs, nymphs and larvae. It has also been found to be effective for the control of certain plant diseases such as citrus greasy spot, helminthesporium and powdery mildew. It has also been a surprising and unexpected discovery that the fish oil component of the present invention greatly reduces or eliminates the phytotoxic effects of certain plant oils such as essential oils.

### SUMMARY OF THE INVENTION

This invention relates to a method of treating plants or habitat thereof, with a composition of plant oil, essential oil and fish oil or mixtures thereof in amounts to effectively mitigate insects and diseases of plants. The composition exhibits a synergistic action in the control of insects and diseases of plants. The plant oils are lighter, less dense and have a higher viscosity than fish oils and provide a quicker mitigation of insects and diseases of plants. The fish oils are heavier, more dense and have a lower viscosity than plant oils and provide a longer residual control of plant insects and diseases. The invention can also be combined with other pesticides to enhance their efficacy. The fish oils in the invention also reduces or eliminates the toxic effects to plants from plant essential oils such as citrus oil or limonene. The composition is preferably applied as a spray, mist or drench to the plants of habitats thereof.

It is an object of the present invention to provide a method of killing and otherwise controlling plant insects and diseases with a pesticide whose active ingredients are derived from naturally occurring components that are safe for the environment and to those applying the pesticide.

It is an object of the present invention to provide a synergistic combination of oils derived from plants and fish that exhibits a quick knock-down and kill while at the same time providing a long term control of plant insects and diseases.

It is an object of the present invention to provide control of insects or diseases of plants by encouraging the development of a predator population of insects, bacteria or fungi target pests injurious to plants.

It is another object of the present invention to greatly reduce or eliminate the toxic effect of essential oils on plants by combining the essential oils such as citrus oil or limonene with fish oil.

It is another object of the present invention to provide a natural pesticide composition that when combined with synthetic pesticides can reduce the use rate of said synthetic pesticides without losing efficacy.

Upon further study of the specification and appended claims, further objects and advantages of this invention will become apparent to those skilled in the art.

### DETAILED DESCRIPTION OF THE PREFERRED EMBODIMENTS

It has surprisingly been discovered that a composition of plant and fish oils can be used to kill, control or in other ways mitigate plant insects and diseases that are injurious to crops in ways unknown prior to this invention. In each embodiment of the subject invention, plant oils combined with fish oils constitute a critical component of the invention and impart a synergy unknown prior to this invention.

This invention does not depend upon chemical compounds that are synergized or not found naturally in nature. This invention was developed by studying the synergistic effect of mixing natural oils derived from plants and fish and applying them in an aqueous solution to plants for the control of insects and diseases of plants.

The objective of this invention was to develop a pesticide comprised of naturally occurring components that poses no harm to the environment or to those applying the pesticide. Pesticides of this nature are often referred to as “soft” pesticides as opposed to “hard” pesticides represented by many synthetically developed pesticides. Heretofore, most soft pesticides have been disappointing by their lack of efficacy and effective control of the targeted insect pests. This invention represents a major departure from the generalized perception that soft pesticides are not dependable control agents for the targeted insect pests and are not as effective as hard pesticides. Due to the synergy exhibited by the combination of plant and fish oils, this invention can adequately replace synthetic, hard pesticides without any loss in ability to control targeted insects and diseases of plants.

The present invention can be used to control a broad range of insects and diseases of plants. These targets include, but are not limited to aphids, chinch bugs, citrus rust mites, spider mites, armored and soft scales, mealy bugs, white-flies, leaf-rollers, leaf miner, fungus gnats, nematodes, sharp-shooters and thrips. It has been found to be effective on all life stages of insects including, eggs, nymphs and larvae. It has also been found to be effective for the control of certain plant diseases such as citrus greasy spot, helminthesporium and powdery mildew.

These targets can be controlled or otherwise mitigated on seeds, corms, bulbs, flowers, stems, leaves and fruits of plants including but not limited to oats, barley, wheat or rice cereals; cotton, tobacco, maize, sorghum, hops, peanuts or soybean field crops; blueberry, cranberry, or strawberry small fruits, banana, coffee or cocoa plantations; peaches, almonds or nectarine stone fruits; apple pome fruits; orange, lemon or grapefruit citrus; pecan or avocado trees; grape vineyards; flowers, herbs, shrubs or ornamentals; curcurbits, cole crops, lettuce, spinach, celery, onions, tomato, or asparagus vegetables; or turf.

The method by which the invention control or mitigates inspects of plants is believed to be by means of suffocation and interruption of the respiratory and nervous systems of insects. The method by which the invention controls or mitigates diseases of plants is believed to be by means of interfering with conidial development of fungi and disruption of the bacterial cell wall. The invention provides a quick knock-down and kill of plant insects and diseases from the plant oils and provides long term control of the same from the fish oils. Another method by which the invention controls both insects and diseases is by ecologically altering the insect and microbial population on the plant or habitat thereof until a population of predator insects and microbes predominates and keeps injurious insect and microbial populations at a low, non-threatening level.

The preferred method of application of this invention is to mix with water and apply pre-harvest or post-harvest to the seed, stem, fruit or foliage of plants or habitat thereof as a spray, mist or drench.

Example 1 illustrates the preparation of a typical composition of the invention.

### EXAMPLE 1

| Component | % by Weight |
|---|---|
| Menhaden Fish Oil | 92.0 |
| Sesame Oil | 5.0 |
| Lecithin | 3.0 |

One half of the required fish oil is added to a mixing vessel. The mixer is turned on and is run at 1200 rpm. With the mixer running lecithin is added followed by sesame oil. The remaining fish oil is added to the vessel and the composition is allowed to mix for three hours.

In accordance with the scope of this invention other plant oils and fish oils can be incorporated in the composition.

### EXAMPLE 2

Test for citrus rust mite control.

The composition (hereinafter referred to as organocide) was applied by an air blast sprayer at the rate of 1.4 and 2.8 gallons per acre in 140 gallons of water on Aug. 27, 1999 to citrus for the control of citrus rust mite. For comparison a test was made with 10.0 ounces of Agri-Mek and 5 gallons of petroleum based horticultural oil (hereinafter referred to as the Check) per acre, also in 140 gallons of water. Pre-treatment rust mite population in the Organocide treatment was very high at 90% and in the Check treatment populations were moderate at 20%. One week after treatment the rust mite population was reduced by 50% by both the Organocide and the Check treatments. By the third week and till the end of the test at the end of the 7th week the rust mite population in the Organocide treatment had been reduced to zero while the rust mite population in the Check averaged 10%.

Result: Organocide was effective on a very high population of rust mites and reduced their population by half the first week. By the 3rd week rust mite population levels were down to zero and remained at that level for the duration of the test. The test shows that Organocide can be effectively used to control rust mites and can substitute for synthetic pesticides, in this case Agri-Mek and petroleum based horticultural oil.

It is believed the mode of action of Organocide was suffocation of the rust mites in the first week followed by an ecological alteration of the insect and microbial population on the leaf surface for the duration of the test.

### EXAMPLE 3

Test for citrus greasy spot control.

Treatments consisted of the following amounts of compounds in 250 gallons of water per acre:

| Treatments | | Disease Incidence |
|---|---|---|
| 1. | Untreated control | 0.510 |
| 2. | Organocide 1.0% v/v | 0.435 |
| 3. | Petroleum oil 1.0% v/v | 0.585 |
| 4. | Organocide 1.0% v/v + | 0.010 |
| | 15 grams Copper/gallon | |
| 5. | Petroleum oil 1.0% v/v + | 0.005 |
| | 15 grams Copper/gallon | |
| 6. | Copper 15 grams/gallon | 0.050 |

The data shows Organocide to be slightly better than petroleum oil when neither is combined with copper. When copper is added to Organocide and petroleum oil the disease incidence dramatically decreases for both treatments. Result:

The test shows that Organocide can be effectively used to control greasy spot on citrus and can substitute for a synthetic pesticide, in this case petroleum based horticultural oil.

### EXAMPLE 4

Test for control of nematodes.

The invention was applied to golf course greens at the rates of 1 and 4 gallons in 100 gallons of water per acre. Before treatment and 1 week after treatment soil samples were taken and analyzed for nematode population. Nematodes targeted were root knot,

[image]

ring, sting and lance nematodes. The percent change in populations were tabulated. The invention had the greatest effect on ring, sting and lance nematodes, reducing their populations by 25% to 100%.

Result: The invention can substitute for chemical pesticides for the control of nematodes.

Although this invention has been described in terms of certain preferred embodiments and applications, other embodiments and applications that may be apparent to those of ordinary skill in the art are also within the scope of this invention.

I claim:

1. A method for protecting plants against insect pests and fungal diseases by applying to the seed, stem, fruit or foliage of the plant or its habitat a synergistic composition comprising oils derived from vegetable and fish origins in a form that is not toxic to the plant.

2. The method of claim 1, wherein said vegetable oil is obtained from plant selected from the group consisting of, but not limited to: canola, castor, cedar, cinnamon, citronella, citrus, clove, coconut, corn, cottonseed, garlic, geranium, lemon grass, linseed, mint, neem, olive, peanut, peppermint, rape, rosemary, sesame, soybean and thyme or mixtures thereof.

3. The method of claim 1, wherein said vegetable oil is an essential oil.

4. The method of claim 1, wherein said vegetable oil is not an essential oil.

5. The method of claim 1, wherein said fish oil is obtained from fish selected from the group consisting of, but not limited to: cod, haddock, capelin, squid, hake, shark, halibut, menhaden, sardine, herring, pollack, cuttle, mackerel, sand eel, anchovy, salmon and gadoid or mixtures thereof.

6. The method of claim 1, wherein an emulsifier is employed and is selected form the group consisting of, but not limited to: lecithin or mixtures thereof, alkylpolymethylsiloxane-copolymer; alkyloxypolyethylenoxyethanol, polyoxyethylene sorbitan ester, polyalkyleneoxide polysiloxane, ethoxylated nonyl phenol, ethoxylated octyl phenol, ethoxylated monoglycerides, ethoxylated diglycerides, ethoxylated alcohols, silicon glycol copolymers, sorbitan fatty acid esters, ethoxylated alkyl phenols, ethylene oxide block polymers, polyether polymethylsilozane copolymer, propolyene oxide block polymers or mixtures thereof.

7. The method of claim 3, wherein the fish oil acts as a carrier for the essential oil or mixtures thereof and reduces or eliminates the plant toxic effect from the essential oil.

8. The method of claim 1, wherein the plant oil or mixtures thereof are resent in an amount of about 1.0 percent by weight to about 99.0 percent by weight.

9. The method of claim 1, wherein the fish oil or mixtures thereof are present in an amount of about 1.0 percent to about 99.0 percent by weight.

10. The method of claim 3, wherein the essential oil or mixtures thereof are present in an amount of about 1.0 percent to about 99.0 percent by weight.

11. The method of claim 4, wherein an emulsifier or mixtures thereof is present in an amount of about 0.5 percent by weight to about 25.0 percent by weight.

12. The method of claim 1, wherein said composition is mixed with water and applied pre-harvest or post-harvest to the seed, stem, fruit or foliage of plants or habitat thereof as a spray, mist or drench.

13. The method of claim 1, wherein said composition is mixed with other pesticides.

14. The method of claim 12, wherein the pesticide is copper.

15. The method of claim 5, wherein said composition is mixed with water in a ready-to-use form at the rate of 1.0% to 50.0% v/v.

16. The method of claim 1, wherein the insects are aphids, chinch bugs, citrus rust mites, spider mites, armored and soft scales, mealy bugs, white-flies, leaf-rollers, leaf miner, mole crickets, nematodes, fungus gnats, sharp-shooters and thrips.

17. The method of claim 1, wherein the disease is greasy spot on citrus, downy mildew and helminthesporium.

18. The method of claim 1, whereby the control of insect pests on plants or habitat thereof, is obtained by direct or indirect contact action, systemic action, anti-feeding effects, repellent effects, ovicidal effects or plant physiology effects.

19. The method of claim 1, whereby control of insects or diseases on plants is accomplished by ecologically altering the insect and disease population by increasing predator insects and microbes on the plant leaf, stem, flower, fruit, seed of habitat thereof that are capable of mitigating insects, bacteria and fungi that are injurious to plants.

20. The method of claim 1, wherein the control of greasy spot on citrus or habitat thereof, is obtained by direct or indirect contact action, systemic action, repellent effects, anti-germination effects or plant physiology effects.

21. The method of claim 5, wherein the plants are: oats, barley, wheat or rice cereals; cotton, tobacco, maize, sorghum, hops, peanuts or soybean field crops; blueberry, cranberry, or strawberry small fruits, banana, coffee or cocoa plantations; peaches, almonds or nectarine stone fruits; apple pome fruits; orange, lemon or grapefruit citrus; pecan or avocado trees; grape vineyards; flowers, herbs, shrubs or ornamentals; curcurbits, cole crops, lettuce, spinach, celery, onions, tomato, or asparagus vegetables; or turf.
